(19) United States
(12) Patent Application Publication
(10) Pub. No.: US 2008/0095797 A1
(43) Pub. Date: Apr. 24, 2008

(54) Recombinant Marek's Disease Virus

(75) Inventors: Martin Allday, London (GB); Venogopal Nair, Berkshire (GB); Andrew Brown, Berkshire (GB)

Correspondence Address: BROOKS KUSHMAN P.C., 1000 TOWN CENTER, TWENTY-SECOND FLOOR, SOUTHFIELD, MI, 48075, US

(73) Assignee: IMPERIAL INNOVATIONS LTD., London (GB)

(21) Appl. No.: 11/571,706

(22) PCT Filed: Jul. 8, 2005
(86) PCT No.: PCT/GB05/02712

§ 371 Date: Mar. 17, 2007

(30) Foreign Application Priority Data
Jul. 9, 2004 (GB) 0415437.3

(51) Int. Cl. A61K 39/00; A61P 43/00; C12N 7/00

(52) U.S. Cl. 424/204.1; 424/93.6; 435/235.1

(57) ABSTRACT

The present invention relates to a Marek's disease virus having a mutation in one or both Meq genes thereof such that the virus is non-pathogenic. The present invention also relates to a vaccine comprising such a virus, and the use of the virus in medicine.

The present invention relates to a Marek's disease virus having a mutation in one or both Meq genes thereof such that the virus is non-pathogenic. The present invention also relates to a vaccine comprising such a virus.

Marek's disease (MD) is a common lymphoproliferative and neurotropic disease of poultry caused by the highly contagious α-herpesvirus called Marek's disease virus (MDV). Because of its contagious nature, rapid onset and long-term persistence in both host and environment, MDV is arguably one of the most economically significant pathogens of poultry. More than 5 billion doses of MDV vaccine are used annually in an attempt to control the disease. The pathogenesis of MD is very complex. Infection is via the respiratory route and is shortly followed by a cytolytic infection of mainly B cells in lymphoid organs. Subsequently, activated T cells (largely of the CD4+ve variety) that are recruited to the site of cytolytic infection become latently infected and become transformed. This leads to neoplastic T cell lesions in visceral organs and infiltrating lymphocytes cause oedema in peripheral nerves and produce paralytic symptoms (Biggs, P. M. (1997) Phil. Trans. R. Soc. Lond. B 352, 1951-1962; Venugopal, K. (2000) Res. Vet. Sci. 69, 17-23). Virus is transported also to feather follicle epithelium, the site of a productive infection that allows shedding and horizontal spread. Although MDV is an α-herpesvirus, biologically it more closely resembles the lymphotropic α-herpesviruses such as EBV, KSHV and HVS.

MD is one of the most costly infectious diseases affecting the poultry industry worldwide and vaccination represents the main strategy for its control. Live vaccines used since the early 1970's are the mainstay of control programmes and are generally very effective. The CV 1988 (Rispens) strain is the most widely used (5 billion doses annually). However, there is a continuous evolution of MDV strains towards greater virulence and these can break through control programmes based on less virulent attenuated strains (Biggs, P. M. (1997) Phil. Trans. R. Soc. Lond. B 352, 1951-1962; Venugopal, K. (2000) Res. Vet. Sci. 69, 17-23). This is obviously of great concern and necessitates the development of new, more effective vaccines. The consensus view is that tailor-made, precisely mutagenised and cloned MDV based on the currently virulent strains established in a BAC system are the best hope for the future (Ross, L. J. N. (1999) Trends in Micrbiol. 7, 22-29; Petherbridge, L. et al., (2003) J. Virol. 77, 8712-8718)

There exists a need for a more effective vaccine against Marek's disease in poultry as the Marek's disease virus (MDV) has become increasingly virulent. Currently available vaccines are less effective against the virus as MDV is evolving continuously to break through the immunity induced by vaccines prepared from less virulent strains of MDV.

Accordingly, a first aspect of the invention provides a Marek's disease virus having a mutation in one or both Meq genes thereof such that the virus is non-pathogenic.

The inventors have found that mutating the Meq genes of MDV renders it non-pathogenic. This finding allows the preparation of MDV strains, including virulent strains that are not affected by existing vaccines, which can be used as a vaccine.

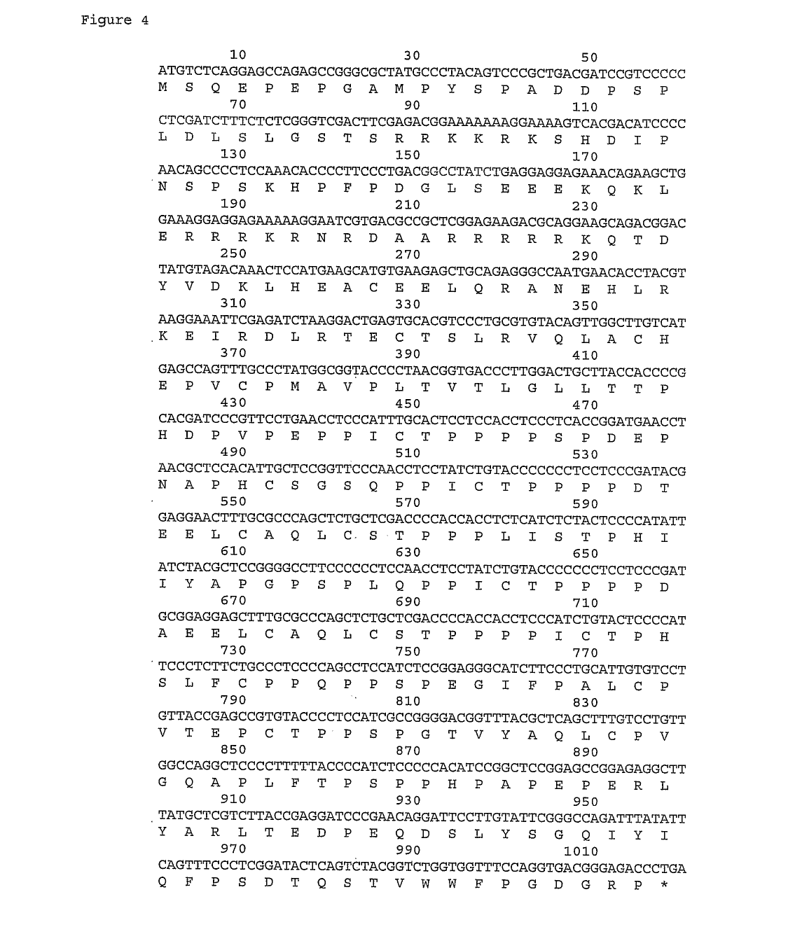

The Meq gene of MDV is considered to be the major transforming oncogene of MDV. There are two copies of the Meq gene in the MDV genome, each encoding a Meq polypeptide, and located in each of two reiterated repeat elements. The nucleic acid sequence of the Meq gene of MDV strain RB-1B and its corresponding amino acid sequence, are shown in FIG. 4 of the accompanying drawings.

The mutation (which may be a deletion, an inversion, an insertion, a substitution, a translocation or any other mutation understood by the skilled person) in one or both of the Meq genes renders the virus non-pathogenic, meaning that it is no longer oncogenic and does not lead to the formation of Marek's disease. However, the virus is still able to replicate in a host cell (in vivo and in vitro) to levels comparable to a wild type pathogenic MDV strain. Previous studies have indicated that MDV replication is important for inducing protection.

In one embodiment of the first aspect, the mutation prevents the polypeptide encoded by the Meq gene binding to a repressor protein of a host cell. A repressor protein is one that is able to inhibit or reduce gene expression. The repressor protein may be C-terminal Binding Protein (CtBP). CTBP has been characterised as a highly conserved repressor of transcription that is involved in cell proliferation. Homologues of CtBP have been identified in mammals, Xenopus and Drosophila. CTBP was identified through its interaction with the C-terminus of the oncogenic E1A protein of adenovirus. The amino acid sequence of the Meq protein contains a conserved bipartite CtBP binding motif, PXDLS motif (₂₀PLDLS₂₄ and ₄₉PDGLS₅₃). The binding motif PXDLS is conserved in many viral and cellular proteins that interact with CtBP.

The inventors have found that mutating the PLDLS motif renders MDV non-pathogenic.

Thus, the mutation may be in a nucleic acid sequence that encodes a PXDLS motif of the polypeptide encoded by the Meq gene. The mutation may be such that the sequence encoding a PXDLS motif is altered or deleted. The PXDLS motif is a highly conserved motif and is required for association of CtBP and the Meq polypeptide. In one embodiment, the mutation is a substitution mutation from P-L-D-L-S to A-V-E-F-T, although those of skill in the art will appreciate that the sequence P-L-D-L-S can be substituted by numerous other sequences. The remainder of the Meq gene sequence may be the same as the wild type Meq gene.

The MDV strain may be RB-1B, a highly virulent highly oncogenic strain of MDV, although it may equally be any other MDV strain. Several strains of MDV are known, including the GA strain (Acc No. NC002229), the Md5 strain (Acc. No. AF243438) or the CV1988 strain, all of which have conserved sequences in this region.

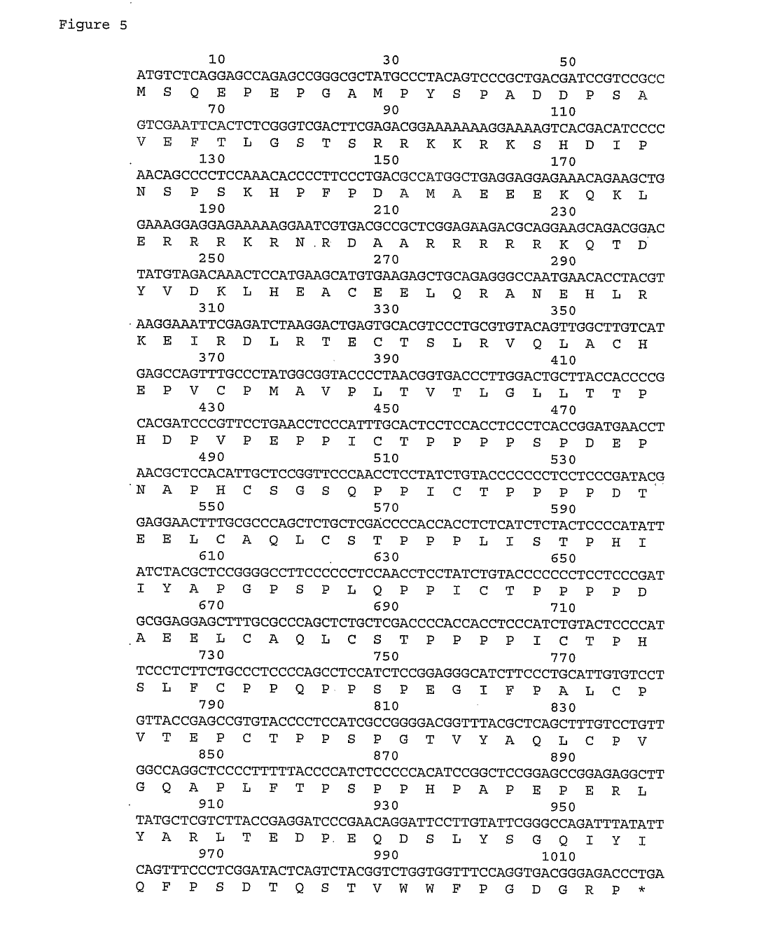

In one embodiment, the mutated Meq gene comprises the sequence shown in FIG. 5 of the accompanying drawings. The skilled person will appreciate that the present invention can encompass variants of this sequence, including, for example, natural strain variations, which may include additions, deletions and or substitutions.

A second aspect of the present invention provides a composition comprising the MDV of the first aspect. The composition may be a vaccine composition, optionally comprising one or more suitable adjuvants. Such a vaccine may be either a prophylactic or a therapeutic vaccine composition.

The vaccine compositions of the present invention can include one or more adjuvants. Examples well known in the art include inorganic gels, such as aluminium hydroxide, and water-in-oil emulsions, such as incomplete Freud's adjuvant. Other useful adjuvants are well known to the skilled person.

A third aspect of the invention relates to a Marek's disease virus of the first aspect, for use in medicine.

A further aspect of the invention provides the use of a Marek's disease of the first aspect in the manufacture of a medicament for the prevention or treatment of Marek' s disease.

The medicament may be for the prevention or treatment of Marek's disease in birds, including poultry, such as chicken, turkey, duck or goose.

In a further aspect, the present invention provides a method for the prevention or treatment of Marek's disease, comprising administering to a subject a therapeutically effective amount of a Marek's disease virus of the first aspect.

Medicaments in accordance with the invention will usually be supplied as part of a sterile, pharmaceutical composition which will normally include a pharmaceutically acceptable carrier. This pharmaceutical composition may be in any suitable form, (depending upon the desired method of administering it to a bird).

It may be provided in unit dosage form, will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, for example by the oral (including buccal or sublingual), topical (including buccal, sublingual or transdermal), or parenteral (including subcutaneous, intramuscular, intravenous or intradermal) route. Such compositions may be prepared by any method known in the art of pharmacy, for example by admixing the active ingredient with the carrier(s) or excipient(s) under sterile conditions.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions)

Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc.

For the preparation of solutions and syrups, excipients which may be used include for example water, polyols and sugars. For the preparation of suspensions oils (e.g. vegetable oils) may be used to provide oil-in-water or water in oil suspensions. Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6): 318(1986).

Pharmaceutical compositions adapted for topical administration may be formulated as ointments, creams, suspensions, lotions, powders, solutions, pastes, gels, sprays, aerosols or oils. For infections of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administration to the eye include eye drops wherein the active ingredient is dissolved or suspended in a suitable carrier, especially an aqueous solvent.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

The pharmaceutical compositions may contain preserving agents, solubilising agents, stabilising agents, wetting agents, emulsifiers, sweeteners, colourants, odourants, salts (substances of the present invention may themselves be provided in the form of a pharmaceutically acceptable salt), buffers, coating agents or antioxidants. They may also contain therapeutically active agents in addition to the substance of the present invention.

Dosages of the substances of the present invention can vary between wide limits, depending upon the disease or disorder to be treated, the condition of the individual to be treated, etc. and a veterinarian will ultimately determine appropriate dosages to be used. The dosage may be repeated as often as appropriate. If side effects develop the amount and/or frequency of the dosage can be reduced, in accordance with normal clinical practice.

Preferred features of each aspect of the invention are as for each of the other aspects mutatis mutandis. The prior art documents mentioned herein are incorporated to the fullest extent permitted by law.

The present invention will now be described with reference to the following non-limiting figures and examples.

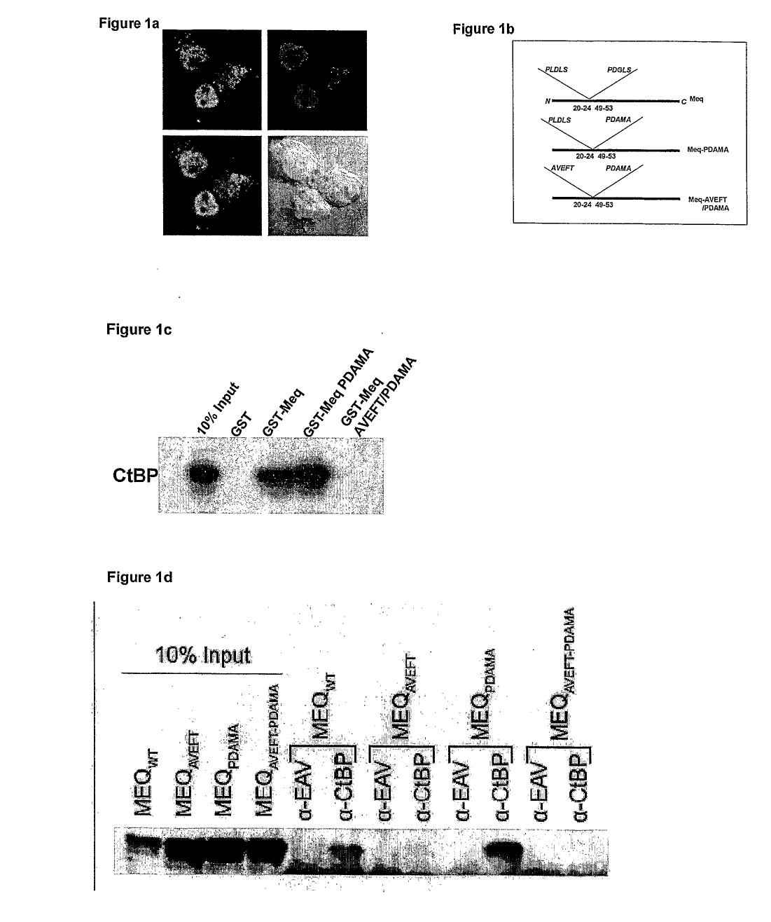

FIG. 1a shows confocal microscopy images showing CtBP and Meq expression in the nucleus of MDV-transformed lymphoblastoid cells. The images show the immunofluorescence test on MSB-1 cell,s showing chCtBP1 (upper left) and MEQ (upper right) and their interaction with the overlay of the two images (lower left). Co-localisation of the two proteins is evident (bottom left). DAPI stained nucleus is also shown (lower right).

FIG. 1b is a schematic view showing the putative CtBP-binding sites in Meq and the mutated sequences.

FIG. 1c shows that when equal amounts of GST or GST-fusions of Meq were incubated with ³⁵S-methionine-labelled CtBP, then separated by SDS-PAGE, and exposed to X-Ray film, CTBP binding is exclusively through amino acids ²⁰PLDLS²⁴. Bacterially expressed GST-MEQ₁₋₁₇₀ was incubated with equal amounts of [³⁵S]methionine-labeled chCtBP1 and the bound proteins were resolved by SDS-PAGE.

FIG. 1d shows the immunoprecipitation of wild type Meq, and Meq with the PDAMA mutation only, with anti-CtBP antibodies. Where the conserved PLDLS sequence had been mutated to AVEFT, Meq was not pulled down. Extracts of DF-1 cells co-transfected with chCtBP1 and MEQ expression constructs were immunoprecipitated with rabbit polyclonal (anti-CtBP or unrelated anti-EAV) antibodies, resolved by SDS-PAGE and western blotted with anti-MEQ antibody.

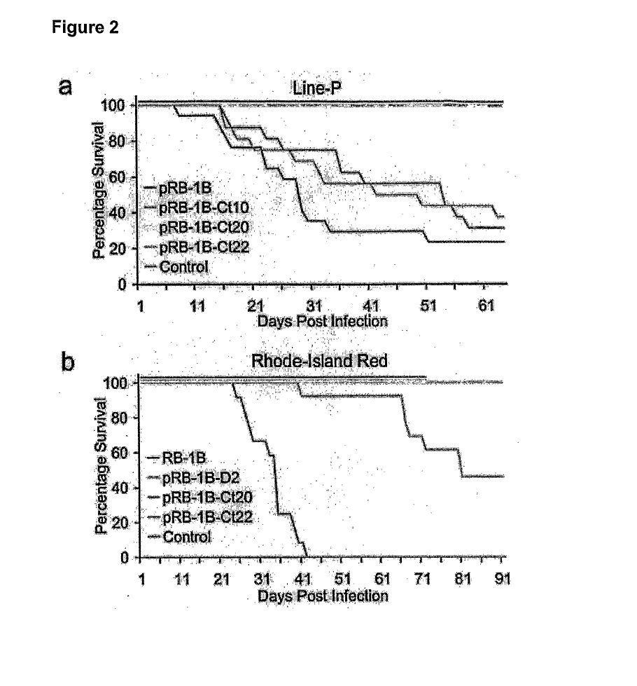

FIG. 2a shows percentage survival curves of line P chickens (1 day old, SPF) infected intra-abdominally with 5000 pfu of each virus stock: wild type RB1B MDV; a mutant with one Meq gene mutated; a mutant with both Meq genes mutated and a revertant [n=15].

FIG. 2b shows percentage survival curves if Rhode Island Red chickens infected with wild type RB1B MDV, a mutant with both Meq genes deleted, a mutant with both Meq genes mutated and a revertent, [n=12.] The pRB-1B-Ct20 virus(both copies of Meq mutated) showed complete loss of oncogenicity in both line-P and RIR lines of birds.

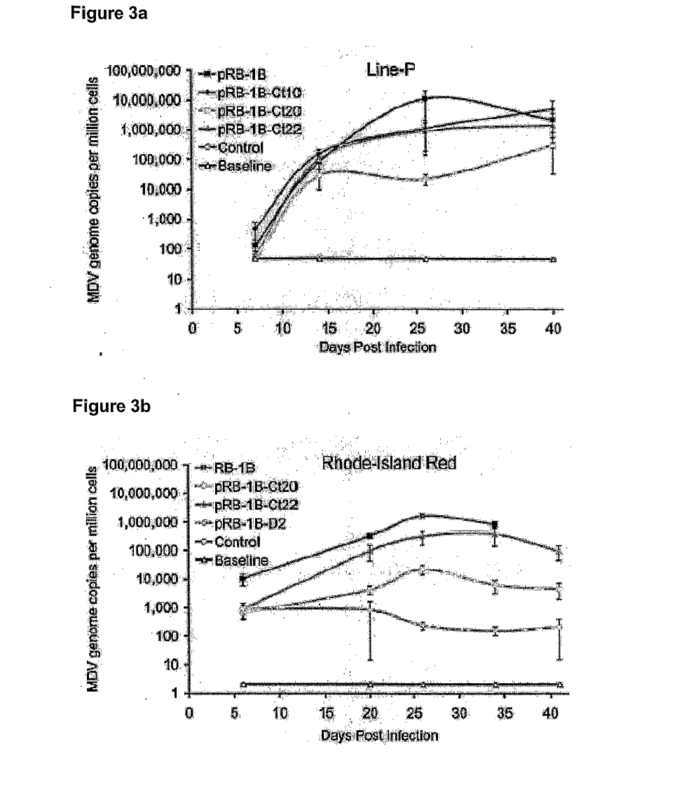

FIG. 3a shows quantitative PCR using primers for the amplification of the MDV Meq gene from peripheral blood leukocytes (PBL) isolated from the chickens in FIG. 2a. Growth curves show evidence of MDV replication in vivo. The TaqMan quantitative PCR tests measured the mean (USE) viral genome copy numbers in PBL of inbred line P chickens (n=6) at 7, 14, 26 and 40 dpi with BAC-derived virus stocks.

FIG. 3b shows quantitative PCR using primers for the amplification of the MDV Meq gene from PBL isolated form the chickens in FIG. 2b. Growth curves again show evidence of MDV replication in vivo. The mean (±SE) viral genome copy numbers in the PBL of outbred RIR chickens (n=6) were measured at 6, 20, 26, 34 and 41 dpi. ΔMEQ deletion mutant pRB-1B-D2) virus was also included in this experiment.

FIG. 4 shows the sequence of the wild type Meq gene of the RB1B strain of MDV.

FIG. 5 shows the sequence of the mutant Meq gene of RB1B strain of MDV having the PLDLS motif thereof mutated to AVEFT.

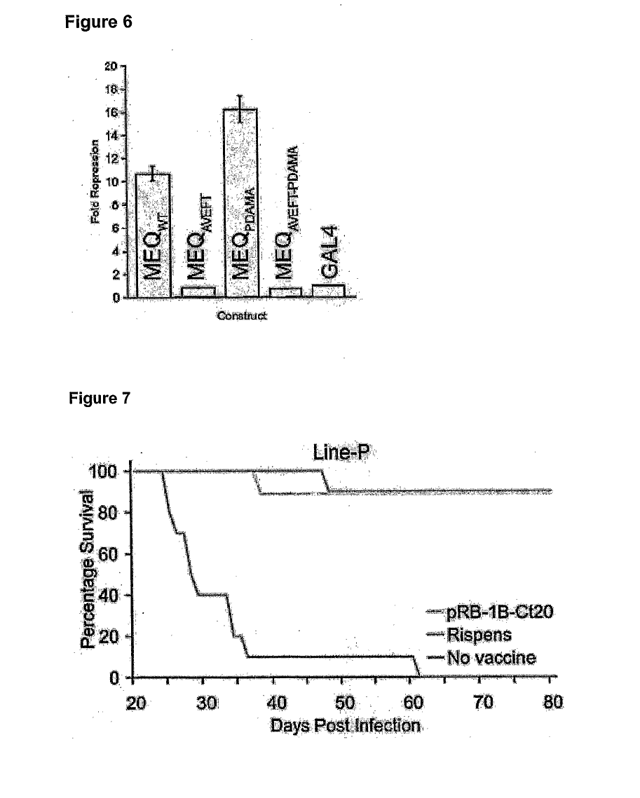

FIG. 6a shows the mean (±SD) luciferase activity (expressed as fold repression over that of the GAL4DBD construct) of extracts of DF-1 cells co-transfected with the firefly luciferase GAL4 reporter, Renilla control plasmid and GAL4DBD-MEQ₁₋₁₇₄ fusion constructs. It can be seen that repression is not seen when the PLDLS sequence of Meq is mutated, indicating that CTBP does not interact with Meq when PLDLS is mutated.

FIG. 7 shows the percentage survival of line P chickens vaccinated with commercial Rispens vaccine or pRB-1B-Ct20 virus (both copies of Meq mutated) [n=9] after challenge with virulent RB1B virus. This figure shows that the mutant MDV successfully prevents 90% of chickens from developing Marek's disease.

### METHODS

### Immunofluorescence Test

MSB-1 cells were fixed in 4% paraformaldehyde for 1 hour and after washing with PBS, they were permeabilised by treating with 0.1% Triton X-100 for 15 min. Washed cells were blocked with PBS+0.5% bovine serum albumen (BSA) for 15 min before incubating with 1:1000 dilution of rabbit anti-human CtBP serum⁵ and 1:100 dilution of mouse anti-MEQ monoclonal antibody FD7 for 1 hour at room temperature. After repeated washing in PBS, cells were then incubated at room temperature with Alexa 488-conjugated goat anti-rabbit- or Alexa 568-conjugated goat anti-mouse antibodies (Molecular probes) for 1 hour. After further washing, cells were stained with DAPI at 1:10,000 and viewed using a Leica confocal microscope.

### GST-Pull Down Assays

These assays were performed essentially as described^{5,15} using in vitro translated [³⁵S]-methionine-labeled proteins. Samples were pre-cleared by absorbing to GST-coated sepharose beads for 1 hour at 4° C. with end-over mixing in 200 μL of EBC buffer (140 mM NaCl, 0.5% Nonidet P-40, 50 mM Tris pH 8.0, 100 mM NaF, 200 μM Na₃VO₄, 1 mg/ml bovine serum albumin). Beads were pelleted by centrifugation and the supernatant incubated with GST or GST fused with the protein of interest for 90 minutes at 4° C. with end-over mixing. The beads were washed extensively with NETN buffer (300 mM NaCl, 1 mM EDTA, 0.5% Nonidet P-40, 20 mM Tris-Cl, pH 8.0) and analysed by SDS-PAGE.

### Immunoprecipitation

Transfected DF-1 cells were washed once with ice-cold PBS and resuspended (1×10⁷ cells/mL) in IP lysis buffer (50 nM Tris-HCl, pH 8.0, 150 mM NaCl, 10% glycerol, 0.5% Triton X-100, 2 mM PMSF) containing proteinase inhibitors (Roche Molecular Biochemicals) for 20 minutes at 4° C. After centrifugation, the supernatant was split into 200 μL aliquots and pre-cleared with 20 μl of protein G-sepharose beads at 4° C. for 1 hour. Beads were pelleted by centrifugation and the supernatant was incubated with rabbit anti-CtBP or the unrelated anti-EAV antibody at 4° C. for 2 hours. Immune complexes were incubated with 30 μL of Protein-G sepharose for 1 hour at 4° C. After washing extensively in IP lysis buffer, the beads were boiled in 30 μL of SDS sample buffer and analysed by SDS-PAGE and western blotting.

### Repression Assay

DF-1 cells co-transfected with expression plasmids of the firefly luciferase GAL4 reporter, Renilla control and GAL4DBD fusion constructs of MEQ. 48 hours after transfection, cells were resuspended in 100 μL of reporter lysis buffer (Promega) and incubated for 15 minutes at room temperature. Cell debris was removed by centrifugation and 20 μL of the clarified lysate was used in luciferase assay. Luciferase activity was measured using the luciferase detection reagent kit (Promega) and expressed as fold repression over that of the GAL4 DBD construct.

### Construction of Mutant Viruses by BAC Mutagenesis

The pRB-1B BAC¹¹ and pST76K_SR construct containing the CtBP binding mutation were electroporated into E. coli DH10B cells and incubated at 30° C. on LB-agar plates containing kanamycin (50 g/ml) and chloramphenicol (30 μg/ml). A few colonies were grown overnight on fresh LB-agar plates at 42° C. Individual colonies were grown on LB medium containing chloramphenicol for 8 hours at 37° C. and then on LB-agar plate containing 10% sucrose at 37° C. overnight. Large-sized colonies were replica plated onto LB-agar containing either chloramphenicol or chloramphenicol and kanamycin and these incubated again overnight at 37° C. Kanamycin-sensitive mutant clones selected by PCR were checked by restriction digestion analysis and growth in transfected CEF. For the construction of BAC clones with mutations in both copies of the MEQ, the process was repeated with the pST76K_SR construct containing the same CtBP binding mutation. A full revertant was constructed by repeating the process twice on the double mutant using the construct containing a wild-type sequence. For further details, see supplementary figure. BAC DNA was transfected into CEF using Lipofectamine (Invitrogen) for the generation of virus stocks.

### Animal Studies

Specific pathogen-free (SPF) inbred line P (B^{21/21}) and outbred Rhode Island Red (RIR) chickens, obtained from the Poultry Production Unit of the Institute for Animal Health, were used. For pathogenicity studies, birds were infected at one-day-old with 5000 pfu of the cell-associated virus stocks by the intra-abdominal route. MDV-infected and the mock-infected control birds were maintained in separate rooms. For the protection study, one-day-old line P birds were vaccinated with 1000 pfu of pRB-1BCt20 or CV1988/Rispens vaccine subcutaneously and challenged with 1000 pfu RB-1B virus at 9 days post-vaccination. The birds that developed the disease and those that survived the experiment were examined at post mortem for any gross/microscopic lesions. All experiments were carried out in accordance with the UK Home Office guidelines.

### EXAMPLE 1

A potential bipartite CtBP-binding motif (₂₀PLDLS₂₄ and ₄₉PGDLS₅₃) in the Meq nuclear protein encoded by MDV was identified. Studies have demonstrated that both human and chicken CtBP bind to Meq both in vitro and in cells through the PLDLS motif located in the amino-terminus of Meq (aa 20-24). Representative results of the confocal microscopy and GST-pull down experiments are shown in FIGS. 1b and 1c. The results of these experiments are indicative of an interaction between CtBP and Meq in vitro. This interaction is entirely dependent on the PXDLS (PLDLS) motif, as indicated by FIG. 1c which shows that there is no binding between MEQ and CtBP when ₂₀PLDLS₂₄ is mutated to ₂₀AVEFT₂₄.

### EXAMPLE 2

In order to determine the significance of the interaction between Meq and CtBP in the pathogenesis of MD and the development of T cell lymphoma, recombinant viruses were generated using the MDV-BAC system based on the virulent RB1B strain of MDV (Petherbridge, L. et al., (2003) J. Virol. 77, 8712-8718). Several mutant viruses were generated including one in which a single copy of Meq was mutated at its CtBP-binding site (PLDLS to AVEFT), one in which both copies of Meq were mutated and a revertent of the virus that had both copies of Meq mutated. Stocks of these viruses were prepared in chicken embryo fibroblasts (indicating that they were all replication competent in vitro) and injected into line-P chickens. The surprising but consistently reproducible result was that while the vast majority of chickens infected with wild type virus, or a single copy mutant or the revertent viruses developed MD with characteristic lymphomas in visceral organs during the 60-day experimental period, in contrast, the control birds and those infected with the double Meq mutant all remained perfectly healthy during the same period of time (FIG. 2a). Rhode Island Red chickens were infected with MDV where both copies of Meq are deleted for PLDLS, a revertent virus, the wild type virus or MDV where both copies of the Meq gene carry the PLDLS to AVEFT mutation (FIG. 2b)

Quantitative-PCR analysis of viral load, performed on peripheral blood leukocytes from birds infected with the wild type and mutant viruses showed similar growth curves (FIGS. 3a and 3b), suggesting that the CtBP-binding mutant virus undergoes cytolytic replication in vivo nearly as efficiently as the wild type MDV. The in vivo growth of wildtype and mutant viruses was examined using a MEQ-specific real-time Taqman quantitative PCR that measures the MDV genome copy numbers in peripheral blood leukocytes (PBL) of experimentally infected chickens. In the first experiment, we used the inbred congenic line P (B^{19/19}) chickens selected for high susceptibility to MD. MDV genome copy numbers in all the groups showed a steady increase during the course of infection with pRB-1B (wildtype), pRB-1BCt10 (one copy of Meq mutated) and pRB-1BCt22(revertent virus) groups registering 1×10⁶ copies per million cells at 40 days post infection (dpi). Birds from the group infected with pRB-1BCt20 (both copies of Meq mutated) carried a lower virus load (3×10⁵ copies per million cells), indicating that these mutations may also have some influence on in vivo MDV replication. However, in experiments in Rhode Island Red (RIR) birds (an outbred population of chickens with a heterogeneous genetic background), viral genome copy numbers (determined using MDV pp38 gene-specific quantitative PCR) of pRB-1BCt20 virus were much higher than those of the MEQ-deletion mutant pRB1B-D2 virus (both copies of Meq deleted), indicating that, compared to the deletion of MEQ, the mutations in the CtBP-binding domain do not completely interfere with virus replication.

### EXAMPLE 3

To establish whether CtBP may be involved in MEQ-mediated transcriptional repression, luciferase reporter assays were performed. The N-terminal 174 amino acids of MEQ were cloned in frame with the GAL4 DNA binding domain. DF-1 cells were co-transfected with a GAL4-responsive luciferase reporter and the fusion constructs of MEQ_{WT}, MEQ_{AVEFT}, MEQ_{PDAMA} or MEQ_{AVEFT-PDAMA} together with Renilla control plasmids. Luciferase assays on the cell lysates showed that both MEQ_{AVEFT} and MEQ_{AVEFT-PDAMA} failed to induce repression, correlating with their inability to interact with CtBP. On the other hand, MEQ_{WT} and the MEQ_{PDAMA} repressed basal transcription from the GAL4 responsive promoter by 10 to 16 fold (FIG. 6), indicating that a transcriptional repression function of MEQ is linked to the ability to interact with CtBP.

### EXAMPLE 4

One day old specific pathogen-free inbred line P chickens were vaccinated with 1000 pfu of pRB-1BCt20 or CV1988/Rispens vaccine subcutaneously and challenged with 1000 pfu RB-1B virus at 9 days post-vaccination. The birds that developed the disease and those that survived the experiment were examined post-mortem for any gross/microscopic lesions. 100% of unvaccinated control birds developed the disease. 90% of those vaccinated with pRB1B-Ct20 virus were protected against virulent MDV infection (FIG. 7). Protection levels were equal to that of the currently used most effective CV1988/Rispens vaccine, demonstrating the successful development of a novel vaccine.

1. A Marek's disease virus having a mutation in one or both Meq genes thereof such that the virus is non-pathogenic.

2. A virus according to claim 1, wherein the mutation prevents the protein encoded by the Meq gene binding to a repressor protein of a host cell.

3. A virus according to claim 2, wherein the repressor protein is CtBP.

4. A virus according to claim 1, wherein the mutation is such that a sequence encoding a PXDLS motif of the protein encoded by the Meq gene is altered or absent.

5. A virus according to claim 4, wherein the PXDLS motif is substituted by another sequence.

6. A virus according to claim 4, wherein the PXDLS motif is PLDLS.

7. A virus according to claim 6, wherein the mutation is P-L-D-L-S to A-V-E-F-T.

8. A virus according to claim 1, wherein the virus is derived from RB-1B strain.

9. A virus according to claim 1, wherein the nucleic acid sequence of each Meq gene comprises the sequence of FIG. 5.

10. A vaccine comprising the virus of claim 1, optionally together with one or more adjuvants.

11. A virus according to claim 1 for use in medicine.

12. The use of a virus according to claim 1 in the manufacture of a medicament for the prevention or treatment of Marek's disease.

13. The use of a virus according to claim 12 for the prevention or treatment of Marek's disease in poultry.
